(19) United States
(12) Patent Application Publication
(10) Pub. No.: US 2022/0136941 A1
(43) Pub. Date: May 5, 2022

(54) METHOD AND APPARATUS FOR THE RAPID DETECTION OF AIR-BORNE VIRUSES

(71) Applicant: Iontrap LLC, Wilmington, DC (US)

(72) Inventor: Anthony Jenkins, North Reading, MA (US)

(21) Appl. No.: 17/516,207

(22) Filed: Nov. 1, 2021

Related U.S. Application Data
(60) Provisional application No. 63/108,471, filed on Nov. 2, 2020.

(51) Int. Cl. G01N 1/44; G01N 27/622; A61B 5/01

(52) U.S. Cl. CPC G01N 1/44; G01K 13/00; A61B 5/01; G01N 27/622

(57) ABSTRACT

Systems for processing a sample are disclosed. The systems include an inlet for receiving the sample comprising target molecules, a filter in fluid communication with the inlet, and an outlet in fluid communication with the filter. The filter is configured to break down the target molecules in the sample and produce breakdown products. The outlet is configured to deliver the breakdown products to a detector.

### CROSS-REFERENCE TO RELATED APPLICATIONS

This application claims priority under 35 U.S.C. § 119(e) to U.S. Provisional Patent Application Ser. No. 63/108,471, titled “METHOD AND APPARATUS FOR THE RAPID DETECTION OF AIR-BORNE VIRUSES,” filed on Nov. 2, 2020, which is incorporated herein by reference in its entirety for all purposes.

### SUMMARY

In accordance with certain aspects, a system configured to process a sample is provided. the system may comprise an inlet configured to receive the sample comprising target molecules. The system may comprise a catalytic filter in fluid communication with the inlet, the catalytic filter being configured to break down the target molecules in the sample and produce breakdown products in a carrier gas. The system may comprise an outlet in fluid communication with the catalytic filter, the outlet being configured to deliver the breakdown products to a detector.

In some embodiments, the catalytic filter includes metallic filaments.

In some embodiments, the metallic filaments are temperature controlled.

The system may further comprise a membrane disposed between the catalytic filter and the outlet.

In some embodiments, the carrier gas comprises nitrogen.

In some embodiments, the carrier gas is air.

The system may further comprise a bypass in fluid communication with the inlet, with a first portion of the sample being delivered to the catalytic filter and a second portion of the sample being delivered to the bypass.

In some embodiments, the first portion of the sample delivered to the catalytic filter is fluidly connected to the bypass downstream from the catalytic filter to produce a combined flow.

In some embodiments, the combined flow is fluidly connected to a second filter and a pump configured to deliver a filtrate of the combined flow to atmosphere.

In some embodiments, the bypass further includes a restrictor configured to control a ratio of the first portion of the sample to the second portion of the sample. The system may further comprise a hood configured to direct the sample to the inlet and prevent the target molecules from escaping to atmosphere.

The system may further comprise a temperature sensor to measure a temperature of a person providing the sample.

The system may further comprise a microphone operably connected to the detector, the microphone configured to relay a processing start time to the detector responsive to a captured sound.

In some embodiments, the detector is configured to collect spectral data from the breakdown products over several seconds and produce a detector signal output.

The system may further comprise a processor operably connected to the detector signal output, the processor comprising a memory storage device and configured to apply the collected spectral data to an artificial neural network trained on a first set of historical spectral data produced by samples known to have a detectable concentration of the target molecules and a second set of historical spectral data produced by samples known to have a non-detectable concentration of the target molecules to produce a result.

In some embodiments, the spectral data includes one or more parameter for each peak selected from peak position, peak size, ratio of peak size to a reference peak size, drift time, appearance time, and change of peak size over time. The memory storage device may be configured to record the spectral data.

In accordance with another aspect, there is provided a system configured to process a sample. The system may comprise an inlet configured to receive the sample comprising target molecules. The system may comprise a filter in fluid communication with the inlet, the filter being configured to break down the target molecules in the sample and produce breakdown products. The system may comprise an outlet in fluid communication with the filter, the outlet being configured to deliver the breakdown products to a detector. In some embodiments, the filter is a thin, perforated metal foil mounted on a metal ring.

In some embodiments, the filter is assembled onto a heated block.

The system may further comprise a ceramic disc mounted on the heated block, the ceramic disc positioned and arranged to form a nominal seal onto the heated block and press the filter onto the heated block.

In some embodiments, the ceramic disc comprises radial grooves contacting the filter and the heated block, the radial grooves positioned and arranged to provide radial flow of hot dry air across the filter.

In some embodiments, the heated block comprises a shallow cavity positioned adjacent the filter dimensioned to allow hot dry air flowing through the filter to pass onto the detector.

The system may further comprise a bypass in fluid communication with the inlet, with a first portion of the sample being delivered to the filter and a second portion of the sample being delivered to the bypass.

In some embodiments, the first portion of the sample delivered to the filter is fluidly connected to the bypass downstream from the filter to produce a combined flow.

In some embodiments, the combined flow is fluidly connected to a second filter and a pump configured to deliver a filtrate of the combined flow to atmosphere.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic drawing of a system for processing a sample, according to one embodiment;

FIG. 2 is a schematic drawing of an input system for an ion mobility spectrometer, according to one embodiment;

FIG. 3 is a schematic drawing of a system for processing a sample, according to one embodiment;

FIG. 4 is a schematic drawing of an input system for a system for processing a sample, according to one embodiment;

FIG. 5 is a schematic drawing of a catalyst filter for processing a sample, according to one embodiment;

FIG. 6 is a schematic drawing of an input system for a system for processing a sample, according to one embodiment; and

FIG. 7 is a schematic drawing of a ceramic disc for providing a radial flow of hot dry air, according to one embodiment.

### DETAILED DESCRIPTION

The recent pandemic caused by the COVID-19 virus has led to the development of detection systems that yield results in as little as a few minutes. Unfortunately, this is not sufficiently rapid to screen large numbers of people entering airports or sports stadia, or even employees entering their place of work. The present disclosure seeks to provide an apparatus for the detection of viruses in six seconds or less, without the need of a clinical staff operator. This will allow rapid screening of subjects at rates of 600 per hour or more.

Prior detection systems have been constructed to detect airborne vapors from contraband such as narcotics and explosives at extremely high sensitivities even below picogram levels of target substances. These detection systems have all relied on the powerful detection capability of Ion Mobility Spectrometers (IMS) or Ion Trap Mobility Spectrometers (ITMS), (collectively, Ion Mobility Spectrometers). Examples of Ion Mobility Spectrometers are shown in U.S. Pat. Nos. 3,699,333 and 5,027,643, incorporated herein by reference in their entireties for all purposes.

Ion mobility spectrometry generally involves analytes being carried into an ionization chamber. A radioactive source, such as ⁶³Ni, can be used to ionize the analyte molecules. The ionized molecules are caused to move down a drift chamber in the detector under the influence of an electric field and are collected on a collector electrode forming a voltage signal over time. The voltage signal is a function of several characteristics of the ion including, for example, size, shape, and charge. The time of flight in the drift tube, also referred to as drift time, can be used to produce a drift spectrum for identification of compounds. Plotting the position and intensity of each value produces a peak for each compound in the sample. A spectrum of peaks is produced many times a second. Taken together, this data may be collectively referred to as spectral data. The spectral data recorded for each scan may include peak size and peak position for every peak in the spectrum. Derivative data may also be recorded for ratios of certain peaks in the spectrum together with change of peak size with time from the start of the test.

Typically, these detectors can detect and identify ions produced by molecules in a size range from 30 to 400 AMU. Unfortunately, a typical virus is many times larger than this, and would not be allowed to pass through the inlet of the detection system, and even if this could be arranged, the size of the subsequent ion would not allow for any resolution in the drift spectrum.

The disclosure seeks to detect target molecules from a sampled stream of air by first causing the target molecules to breakdown on a filter to produce fragments within a detectable size range, for example, a size range from 30 to 400 AMU. In certain embodiments, the disclosure provides methods and systems for detecting viruses from the sample by thermally breaking down the virus on a hot catalyst grid or filter placed in the airstream entering the detection system. The COVID-19 capsid consists of an envelope surrounded by many spike proteins distributed around it. The aim of the disclosure is to cause the breakdown of the spike proteins into smaller amino acid fragments that can then be detected in an ion mobility spectrometer based system. The ITMS, in particular, is selectively sensitive to amino compounds that can be detected in the positive ion mode. Exemplary ITMS detection is detailed in U.S. Pat. No. 5,491,337, incorporated herein by reference in its entirety for all purposes.

In order to improve probability of detection, it is envisaged that each subject is asked to cough into the intake of the detection system. Since this will cause the emission of viruses from infected persons, the intake is designed to capture the whole volume of the breath and draw it into the detection system where a sample of the breath is directed to a filter of the system, for example, caused to impinge on a hot catalytic surface. The remainder of the sampled air bypasses the filter. The sample directed to the filter is caused to rejoin the bypass flow downstream from the filter to produce a combined flow. The combined flow is directed to an exhaust comprising a disinfecting filter to remove any virus before exhausting back into the atmosphere.

As a further precaution that will supplement the possibility of identifying subjects who have late stage infection when the emission of viruses may be reduced, a system and method for remotely detecting the skin temperature of the subject is included. Alarm outputs may be provided from one or both the vapor detector and the temperature test. The temperature test will not be specific to any particular virus, but the ion mobility test will be more specific.

It is possible that a small level of false positives to the particular COVID-19 virus may be produced. These can be confirmed by a conventional clinical test that may take several minutes, but since the incidence of such alarms will be small, this will not unduly affect the throughput rate.

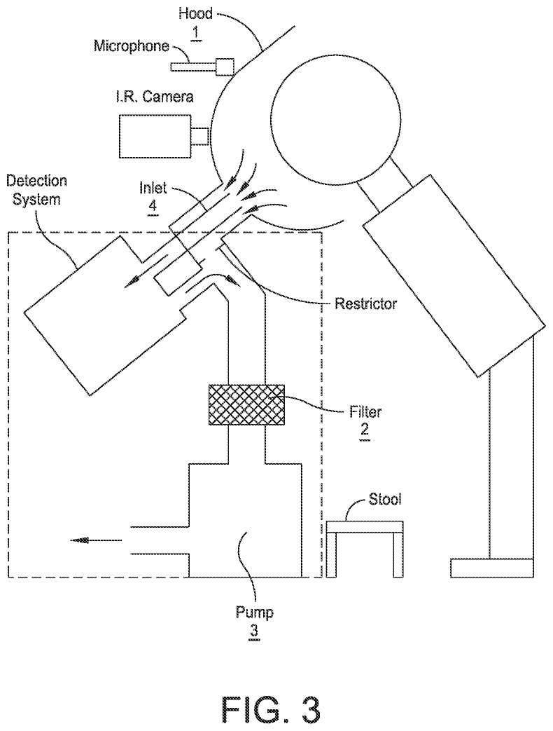

In some embodiments, an inspection station for the rapid detection of viruses expelled from the mouth of a subject is constructed as shown in FIG. 3. A plastic hood 1 is arranged at an angle to the vertical so individuals of varying heights from small youths to tall adults can access the hood as shown in FIG. 3. For very small children, a drop-down stepstool may be provided attached to the front of the test station. The hood may be transparent or any color. The hood may be plastic or any suitable material. An alternate arrangement allows for the hood 1 to be made to move vertically to the correct position for the test. The movement may be performed in response to the measurement of the subject's height. An optional infrared camera may be placed to view the area of the subject's head. The radiated infrared from the subject's forehead is focused onto an infrared sensor imaging array. The low resolution image is then subjected to image analysis to detect the hot spots in the image. When the image, for example, four or more contiguous pixels, indicate that normal skin temperature has been exceeded, a temperature alarm is made.

To begin the virus test, the subject coughs into the center of the hood 1 where an exit port draws in all the breath into the system by the action of an air pump 3. An optional microphone may be placed to capture the coughing sound. A disinfecting filter 2 is arranged before the pump 3 to remove any contagious viruses before exhausting back into the atmosphere. Optionally, the hood may include one or more orifices. The orifices may generally be small holes positioned in the hood around the inlet tube. The orifices may provide a bypass flow path for directing a portion of the breath to atmosphere. All sampled air is passed through the disinfecting filter prior to being directed to atmosphere. A portion of the sampled air is directed to the detection system. Downstream from the detection system, the portion of the sampled air is directed to the bypass, producing a combined flow. The combined flow is directed to the filter 2 before being pumped to atmosphere.

The portion of the sampled air that is fed into the detection system is directed to a catalytic filter upstream of the detection system, where target molecules, for example, virus capsids, are caused to breakdown into fragments or breakdown products that are then carried in a carrier gas to the detector, such as an ion mobility spectrometer, where they may be detected and identified. The carrier gas may comprise nitrogen, optionally the carrier gas may be air. In certain embodiments, the carrier gas is temperature controlled, for example, heated, such that it is in the form of hot dry air.

An optional microphone may be positioned to capture a sound, such as the coughing sound from the subject. The microphone may be operatively connected to the detection system to accurately relay start time of the test.

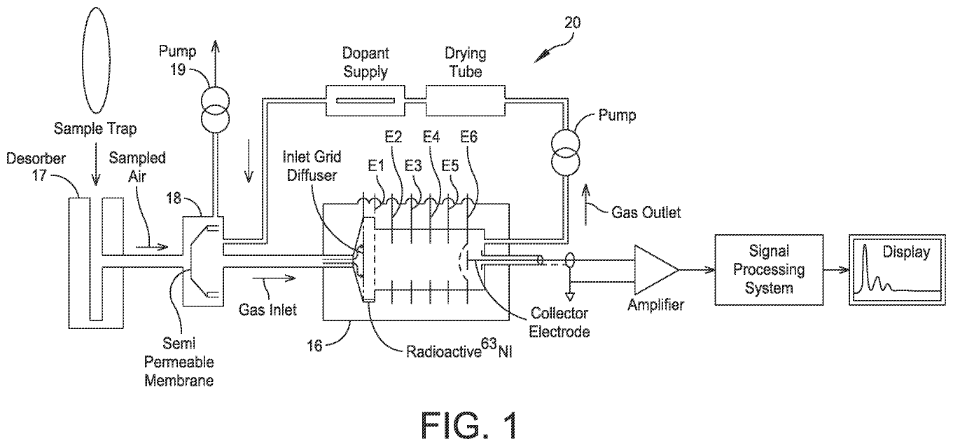

Ion mobility detection systems are described in U.S. Pat. Nos. 5,491,337 and 7,942,033, incorporated herein by reference in their entireties for all purposes. FIG. 1 shows a system 20 in which sample was collected on a filter paper, or similar material, then evaporated in a desorber 17, before being drawn into the detector 16 by sampling pump 19. Ion mobility spectrometers are negatively affected by water vapor and other atmospheric contaminants. It is necessary to exclude such contaminants before being allowed into the detector 16 itself. One way this may be achieved is to use a semi permeable membrane 18 in the input to the detector 16 as shown in FIG. 1. This exemplary membrane 18 comprises dimethyl silicone which is particularly permeable for organic molecules in a size range up to about 400 AMU. The membrane 18 is largely impervious to inorganic molecules, including water vapor.

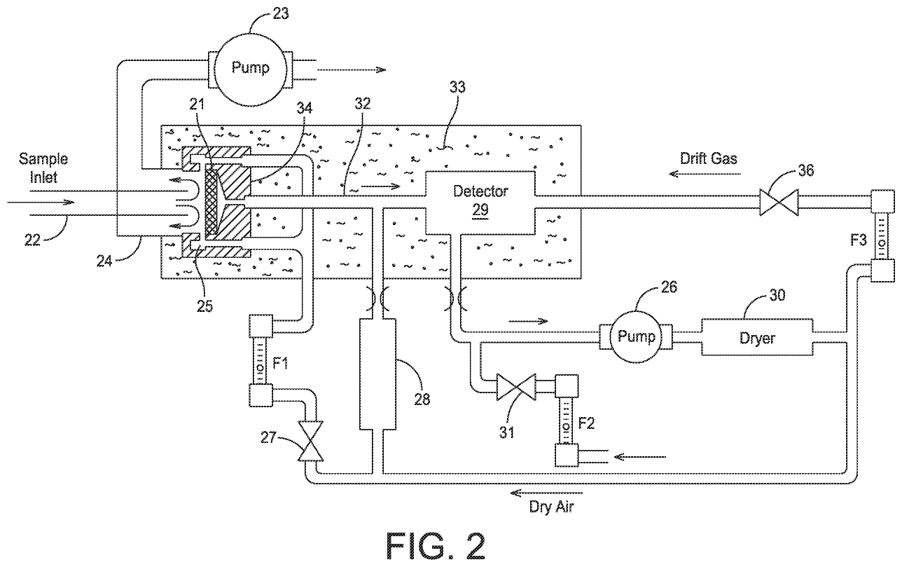

An alternate input system for an ion mobility spectrometer is shown in FIG. 2. This system is designed to allow the passage of inorganic molecules, such as, peroxide explosives, that do not readily pass through the dimethyl silicone membrane 18 shown in FIG. 1. Instead, the system of FIG. 2 includes microporous filter element 21. As shown in the system of FIG. 2, sampled air is drawn in through an inlet tube 22 by the action of a pump 23. Sampled air passing down the inlet tube 22 impinges on the porous filter element and turns back through the concentric tube 24 before continuing on to the pump 23. Dry air is injected into a boundary region through an array of jets 25 arranged in a ring at the surface of the filter element 21 by the action of a pump 26. Dry air curtain may be pre heated in housing 34 The filter housing, connecting tube 23, and detector 29 may all be maintained at an elevated temperature and insulated by thermal insulation material 33.

The air passing through the filter element 21 is drawn through the detector 29 then to the pump 26 and onto drying system 30 before being directed to several dry air flows. The volume of the filter element 21 and connecting pipe 32 to the detector 29 is kept small so that the transit time from the filter 21 to detector 29 is less than one second. A small flow of dry air is provided through a dopant chamber 28. Valve 31 controls make up air flow intake into the vacuum side of pump 26. Valve 36 controls drift gas into detector 29. Valve 27 controls dry air flow to dry air curtain 25. F1, F2, and F3 are flow meters.

The COVID-19 capsid in its entirety will not pass through either of these two input systems shown in FIGS. 1 and 2, nor will the capsid in its entirety itself produce a viable spectrum in the detector since it is much too large to pass down the ion drift region and provide resolvable spectra. However, portions of the capsid may pass through the disclosed inlet systems.

Thus, the disclosure provides systems and methods that break down the COVID-19 capsid into viable amino acid fragments that will pass through each type of inlet system to an Ion Mobility Spectrometer. The fragments may be detected in both the positive and negative ion modes of detection.

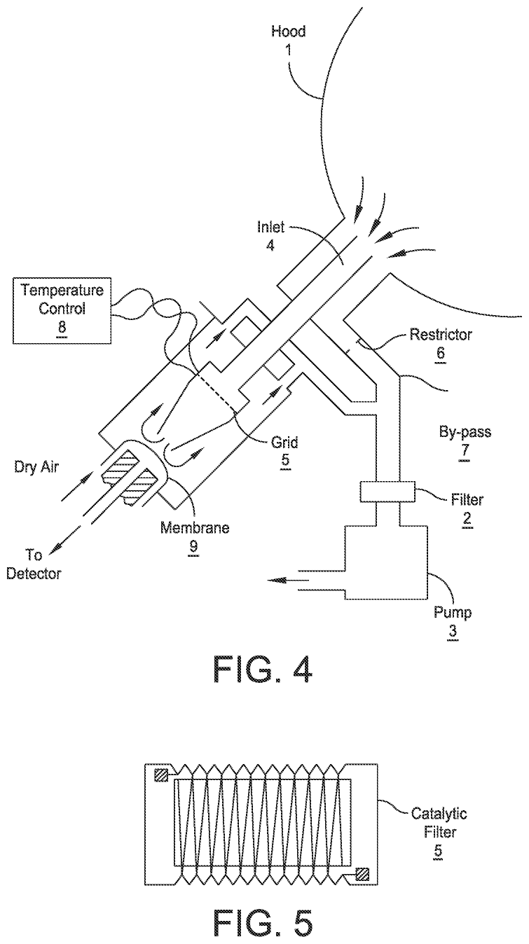

One embodiment of the modified inlet system is shown in FIG. 4. Air is drawn from within the hood 1 by the action of a pump 3. All the air drawn from the hood 1 is passed through a filter 2 to remove and destroy contagious viruses. Some of the sampled air is drawn through the inlet tube 4 and is caused to flow through a catalytic filter 5 comprising a temperature controlled heated platinum filament grid. Here, any viruses in the air stream are broken into fragments that include amino fragments from the breakdown of spike proteins in the viral capsid. Other metallic filaments may be employed to break down the virus capsid. A platinum wire filament grid is one exemplary embodiment that is reliable in producing detectable fragments. The breakdown products are carried on the air stream and are caused to impinge on the dimethyl silicone membrane of a detection system. The amino acid fragments pass readily through the membrane and are quickly detected and identified by the ion mobility spectrum produced.

After impinging on the dimethyl silicone membrane, the sampled air flow is directed to join the bypass air stream as shown in FIG. 4, where it passes through the disinfecting filter 2, and on to the pump 3. The proportion of air passing through the catalyst versus the bypassed air flow is controlled by a restriction 6 in the bypass line 7. The bypass air stream allows the hood 1 to be quickly and completely vented without forcing all the sampled air through the catalyst grid. The sampled air passing through the catalyst is drawn from the center region of the stream of air leaving the hood where the concentration of virus capsids is generally greater. The air pump 3 is designed for continuous operation, maintaining the air flow through the hood at all times to prevent virus from escaping into the atmosphere. If or when a positive viral presence is detected, the hood may be disinfected by spraying with alcohol or another agent that will kill the virus. The disinfecting agent may also kill or sterilize any virus trapped in the exhaust filter.

The design of one embodiment of the platinum catalyst is shown in FIG. 5. The catalyst filter 5 comprises a molded ceramic plate shown in plan view in FIG. 5. The exemplary ceramic plate is a rectangular molded piece with a hollow center, and grooves on each side to allow platinum wire to be wound into a grid pattern as shown in FIG. 5. The platinum wire grid is connected to a power supply and temperature control system 8 of FIG. 4. The platinum wire temperature is measured by determining the resistance of the wire as is commonly employed in temperature control systems using a platinum resistance thermometer. The resistance of the wire increases as the temperature increases, thus allowing accurate control and ability to set varying temperatures of the wire itself. The increased temperature of the platinum wire also heats the sampled air flow that then impinges on the membrane window 9 shown in FIG. 4. The platinum wire temperature is set at a value that enables or optimizes both the catalytic breakdown and subsequent diffusion through the di-methyl silicone window. The diffusion rate through the membrane window normally increases with increased temperature but is controlled to not exceed the maximum operating temperature of the dimethyl silicone membrane material.

During operation, the proteins of the viral capsid of COVID-19 are caused to breakdown into constituent amino acid fragments on the catalytic surface of the inlet systems described herein. The constituent amino acids may themselves be further fragmented on the catalytic surface, forming smaller fragments, several comprising an amine group (—NH₂). Such fragments are strongly electropositive, and readily form positive ions in the ionization chamber of the ion mobility spectrometer. The fragments generally include several different sizes and chemical structures and are detected as separate peaks in the positive ion mobility spectrum. Some fragments, however, may produce negative ions and are detected in the negative ion mode of an ion mobility detector.

Unfortunately, other proteins present in the nose or throat of a subject, such as those in mucus, when expelled into the inlet of the detection system, also breakdown and form a spectrum in both positive and negative modes of the ion mobility detector. Some of the spectral peaks may be identical to those produced by COVID-19 viral capsids. No one peak is unique to the COVID-19 spectral response. When spectra are produced from several types of protein, they become very complex and the response to COVID-19 capsids may be masked. Thus, in some embodiments, more parameters in the response profile may be measured in order to make a successful detection diagnosis, reducing the incidence of false positives and false negatives.

Since it is not permissible to routinely test the equipment on a live COVID-19 sample, an alternate, non-contagious sample such as that from a plant virus can be used to test the system on a routine basis. The spectrum produced by the inactive virus will be different to that produced by the COVID-19 virus but is a safe way of checking and calibrating correct operation of the system. The system may additionally be self-calibrating with data from calibration samples, optionally with data from tested samples. Data from the ion mobility detector may be obtained and stored in a memory storing device. A processor may be coupled to the memory storing device. This data includes spectral peak positions in both positive and negative spectra, sizes of each peak in the spectrum, ratios of peak sizes, appearance time and duration of each peak, ratios of peaks with time, and other parameters.

In order to uniquely identify positive COVID-19 samples, the spectral data produced from tests on a known population of COVID and non-COVID samples (e.g., data produced every 20 milliseconds or less), may be collected. The data may include one or more of spectral peak positions in both positive and negative ion modes of detection, the size of each peak, and the appearance time and duration of each peak. The data may be processed to obtain derivative data. Derivative data may then be recorded, providing ratios of peaks in the spectra, variation of these ratios with time, and change of peak size with time from the start of the test. The data set from each test may be fed into a neural network that has been previously trained on a known population data set. The neural network may be used to rapidly obtain and provide a yes or no answer for the presence of the COVID-19 virus in the sample.

Once trained, the parameters of the neural network may remain set or be re-trained periodically. An alarm output identifying a sample as detecting COVID-19 or not detecting COVID-19 (e.g., “clear” or “not detected”) may be provided. No knowledge of the chemical composition of the viral fragments is required, and the process may be considered analogous to detection of contraband by trained K-9 units, that alarm only on the substance they have been trained to detect. Other infections of the nose and throat can be similarly detected after training on a relevant population. Accordingly, while the disclosure contemplates detection of the COVID-19 virus, it should be understood that similar systems and methods may be used to detect other viruses or other analytes, such as other microorganisms, that are generally too large for detection with conventional systems.

The flow of sample from the hood through the catalyst and the membrane window and the subsequent analysis in the detector may take less than one second, but the sensitivity of the ion mobility detector is improved by averaging the spectra over a period of a few seconds. Multiple measurements from the same sample may be collected over this period of time. Thus, the total analysis time from cough to detection result can be set from one to five seconds. Only if a positive alarm is produced is it necessary to decontaminate the hood with a disinfectant spray or wipe.

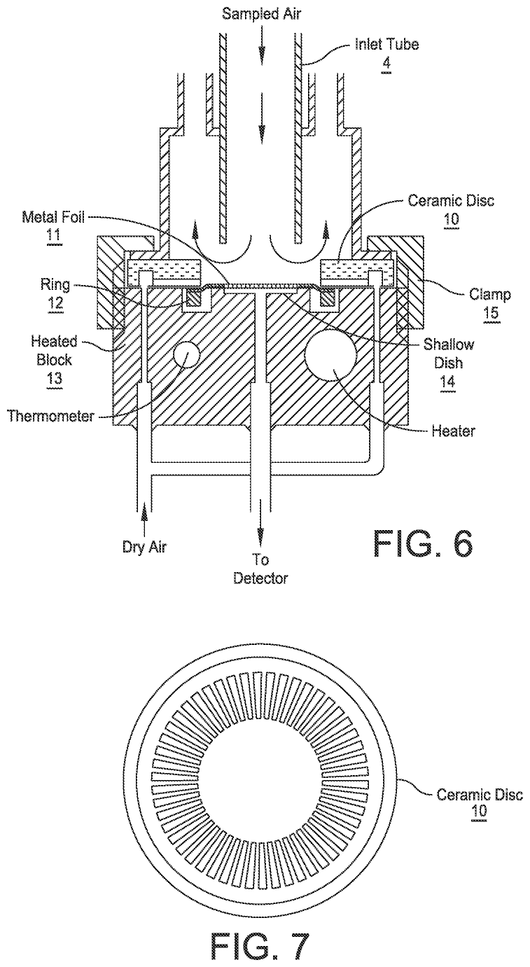

The input system to an ion mobility detector as described in U.S. Pat. No. 7,942,033 allows a different catalyst arrangement. The principle however, remains the same as previously described herein. As shown in FIG. 2 the input air flow is arranged to impinge onto a heated filter 21, where sample is drawn through the filter 21 by the reduced pressure across the filter element produced by the pump 26, which is connected to the outlet of the detector 29. In order to allow the catalytic breakdown of the COVID-19 virus and subsequent detection of the fragments, the filter 21 may be modified as shown in FIG. 6.

The exemplary embodiment of FIG. 6 includes a thin metal foil 11, for example, made of stainless steel, copper, or any suitable material mounted on a metal ring 12. The thin metal foil 11 may be mounted by electron beam welding of the foil to the ring. Stainless steel foil can only be welded to a stainless steel ring and similarly copper foil can be electron beam welded to a copper ring. Sub-micron size holes may be formed by laser drilling of the foil to provide a pervious filter as shown in FIG. 6. The filter area may be arranged to be approximately the same diameter as the inner diameter of the sample inlet tube 4 (for example, as shown in FIG. 3). When copper foil is employed, it is preferable to protect it by plating with a suitable material, such as nickel, for example. Finally, the filter area may be, but not necessarily, covered with a thin layer of platinum by either electro plating or vapor deposition.

The filter may be assembled into the heated block 13, which is illustrated in FIG. 6. A feature of this assembly is the formation of a boundary layer of hot dry air over the filter, for example, as described in U.S. Pat. No. 7,942,033. The incoming air stream, which acts as the carrier gas, is directed onto this layer. Only heavy molecules and micro-organisms are allowed to penetrate into the hot boundary layer by setting the length of the inlet tube 4 to the point where heavy molecules are allowed through by virtue of their greater momentum. Thus, many pollutants that would normally be detected may be prevented from entering the detector. The dry air boundary layer is supplied from a ring above the filter that is connected to a multiplicity of radial grooves formed in a ceramic disc 10, which is shown to be assembled into the inlet system in FIG. 6, and in further detail in FIG. 7. The ceramic ring is clamped onto the heated block 13 forming a nominal seal onto the block, and at the same time, pressing the filter onto the block. The filter is depressed into the heated block 13 by the thickness of the metal foil 11. This ensures good thermal contact onto the heated block 13 and stretches the foil tight. The area below the filter region is machined in a shallow circular dish 14 to allow the air flowing through the filter to pass onto the detector input. The catalytic filter 5 (for example, as shown in FIGS. 4 and 5) may be maintained at a temperature close to that of the heated block 13. Thus, the depth of the dish 14 may be designed to ensure good heating of the catalyst while still allowing the air to flow to the detector. The depth of the dish 14 may be, for example, less than one millimeter.

It is envisaged that the catalytic filter 5 may need to be replaced occasionally, so in some embodiments the sample inlet 4 is made detachable from the heated block 13 by an insulating clamp nut 15, which is shown in FIG. 6. The clamp nut 15 may be designed to ensure that the inlet tube 4 is set at the correct distance from the metal foil filter 11.

The block itself is maintained at a regulated temperature that ensures rapid breakdown of a virus on the platinum coated filter. This may be achieved by a cartridge heater inserted into the block as shown in FIG. 6. A thermometer may also be inserted into the block and the temperature is controlled by a conventional temperature control system.

On entering the ion mobility detector, any breakdown products from the catalytic reaction on the hot platinum layer are ionized in the detector and drift spectra in both positive and negative ion modes are continuously obtained. Spectral data may be taken approximately every 20 milliseconds. Multiple measurements of the sample may be repeated for a duration of a few seconds, for example, 2-5 seconds. The data is then passed into the artificial neural network where the analysis of the data is made, as previously described. Any changes affecting the mobility spectrum caused by temperature or pressure changes in the detector can be monitored and corrected by routinely calibrating the equipment with a harmless reference virus sprayed into the hood.

As an added precaution, a temperature test on the subject may be provided concurrent with the virus inspection test. Conventional radiation temperature sensors typically require an operator to hold the thermometer within one or two inches of the subject's forehead. Here, a temperature sensor is designed to operate automatically by scanning the subject using an infrared low resolution camera. It is not necessary to accurately position the forehead since the area of the head is scanned and focused onto a sensor array that covers the whole area of the subject's upper head. The image is analyzed, and the highest temperature is identified. If this exceeds the normal skin temperature of a person in the particular environment of the test, then a temperature alarm is made.

In accordance with certain aspects, there is provided a method of processing a sample. The method may be performed with the systems described herein. The method may generally include receiving a sample from a subject. The sample may be a respiratory sample, for example, a cough. The method may include directing part of the sample to a catalyst filter configured to cause a target molecule, e.g., virus capsid, to breakdown into fragments detectable and identifiable by an ion mobility spectrometer. The method may include selecting the part of the sample directed to the catalyst filter, for example, a target volume of the sample, and directing the remainder of the sample to a bypass. The method may include controlling temperature of the sample or breakdown product. The method may include directing the part of the sample directed to the detector (i.e., any breakdown products) and any residual part of the sample (for example, directed to a bypass) back to atmosphere. The sample and breakdown products directed to atmosphere may first be directed to a disinfecting filter. The method may comprise obtaining spectra from the breakdown products and comparing the spectra to a known profile of a target species, such as COVID-19, optionally with an artificial neural network as previously described. The method may comprise identifying whether the target species is detected or not detected from the breakdown products.

This disclosure is not limited in its application to the details of construction and the arrangement of components set forth in the following description or illustrated in the drawings. The principles set forth in this disclosure are capable of being provided in other embodiments and of being practiced or of being carried out in various ways. Also, the phraseology and terminology used herein is for the purpose of description and should not be regarded as limiting. The use of “including,” “comprising,” “having,” “containing”, “involving”, and variations thereof herein, is meant to encompass the items listed thereafter and equivalents thereof as well as additional items.

Having thus described several aspects of at least one embodiment of this disclosure, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

What is claimed is:

1. A system configured to process a sample, the system comprising:
an inlet configured to receive the sample comprising target molecules;
a catalytic filter in fluid communication with the inlet, the catalytic filter being configured to break down the target molecules in the sample and produce breakdown products in a carrier gas; and
an outlet in fluid communication with the catalytic filter, the outlet being configured to deliver the breakdown products to a detector.

2. The system of claim 1, wherein the catalytic filter includes metallic filaments.

3. The system of claim 2, wherein the metallic filaments are temperature controlled.

4. The system of claim 1, further comprising a membrane disposed between the catalytic filter and the outlet.

5. The system of claim 1, wherein the carrier gas comprises nitrogen.

6. The system of claim 5, wherein the carrier gas is air.

7. The system of claim 1, further comprising a bypass in fluid communication with the inlet, with a first portion of the sample being delivered to the catalytic filter and a second portion of the sample being delivered to the bypass.

8. The system of claim 7, wherein the first portion of the sample delivered to the catalytic filter is fluidly connected to the bypass downstream from the catalytic filter to produce a combined flow.

9. The system of claim 8, wherein the combined flow is fluidly connected to a second filter and a pump configured to deliver a filtrate of the combined flow to atmosphere.

10. The system of claim 7, wherein the bypass further includes a restrictor configured to control a ratio of the first portion of the sample to the second portion of the sample.

11. The system of claim 1, further comprising a hood configured to direct the sample to the inlet and prevent the target molecules from escaping to atmosphere.

12. The system of claim 1, further comprising a temperature sensor to measure a temperature of a person providing the sample.

13. The system of claim 1, further comprising a microphone operably connected to the detector, the microphone configured to relay a processing start time to the detector responsive to a captured sound.

14. The system of claim 1, wherein the detector is configured to collect spectral data from the breakdown products over several seconds and produce a detector signal output.

15. The system of claim 14, further comprising a processor operably connected to the detector signal output, the processor comprising a memory storage device and configured to apply the collected spectral data to an artificial neural network trained on a first set of historical spectral data produced by samples known to have a detectable concentration of the target molecules and a second set of historical spectral data produced by samples known to have a non-detectable concentration of the target molecules to produce a result.

16. The system of claim 15, wherein the spectral data includes one or more parameter for each peak selected from peak position, peak size, ratio of peak size to a reference peak size, drift time, appearance time, and change of peak size over time, and the memory storage device is configured to record the spectral data.

17. A system configured to process a sample, the system comprising:
an inlet configured to receive the sample comprising target molecules;
a filter in fluid communication with the inlet, the filter being configured to break down the target molecules in the sample and produce breakdown products; and
an outlet in fluid communication with the filter, the outlet being configured to deliver the breakdown products to a detector,
wherein the filter is a thin, perforated metal foil mounted on a metal ring.

18. The system of claim 17, wherein the filter is assembled onto a heated block.

19. The system of claim 18, further comprising a ceramic disc mounted on the heated block, the ceramic disc positioned and arranged to form a nominal seal onto the heated block and press the filter onto the heated block.

20. The system of claim 19, wherein the ceramic disc comprises radial grooves contacting the filter and the heated block, the radial grooves positioned and arranged to provide radial flow of hot dry air across the filter.

21. The system of claim 19, wherein the heated block comprises a shallow cavity positioned adjacent the filter dimensioned to allow hot dry air flowing through the filter to pass onto the detector.

22. The system of claim 17, further comprising a bypass in fluid communication with the inlet, with a first portion of the sample being delivered to the filter and a second portion of the sample being delivered to the bypass.

23. The system of claim 22, wherein the first portion of the sample delivered to the filter is fluidly connected to the bypass downstream from the filter to produce a combined flow.

24. The system of claim 23, wherein the combined flow is fluidly connected to a second filter and a pump configured to deliver a filtrate of the combined flow to atmosphere.
